(19) United States
(12) Patent Application Publication
(10) Pub. No.: US 2014/0162891 A1
(43) Pub. Date: Jun. 12, 2014

(54) METHODS AND MATERIALS FOR DETECTION, DIAGNOSIS AND MANAGEMENT OF OVARIAN CANCER

(71) Applicant: UNIVERSITY OF SOUTH FLORIDA, Tampa, FL (US)

(72) Inventors: Rebecca SUTPHEN, Tampa, FL (US); Johnathan Mark LANCASTER, Tampa, FL (US)

(21) Appl. No.: 13/947,944

(22) Filed: Jul. 22, 2013

Related U.S. Application Data
(62) Division of application No. 12/524,537, filed on Mar. 24, 2010, now abandoned.
(60) Provisional application No. 60/886,778, filed on Jan. 26, 2007.

(51) Int. Cl. G01N 33/574

(52) U.S. Cl. CPC G01N 33/57449
USPC 506/9; 435/7.92; 435/7.4; 435/7.94; 435/7.93; 436/501; 436/86

(57) ABSTRACT

The subject invention concerns methods using a panel of proteins to detect, diagnose, and monitor therapy during treatment of ovarian cancer in a female patient. The proteins were identified using proteomics analyses of plasma samples obtained preoperatively from ovarian cancer patients versus those of healthy control women. Such a panel has utility for the diagnosis of ovarian cancer, screening for ovarian cancer and possibly therapeutic monitoring.

### CROSS-REFERENCE TO RELATED APPLICATIONS

The present application is a divisional of U.S. application Ser. No. 12/524,537, filed Mar. 24, 2010, which is the national stage of International Application Number PCT/US2008/052078, filed Jan. 25, 2008, which claims the benefit of U.S. Provisional Application Ser. No. 60/886,778, filed Jan. 26, 2007, each of which is hereby incorporated by reference herein in its entirety, including any figures, tables, nucleic acid sequences, amino acid sequences, and drawings.

### BACKGROUND OF THE INVENTION

According to the American College of Obstetrics and Gynecology, 5-10% of women in the U.S. will undergo a surgical procedure for a suspected ovarian neoplasm during their lifetime. A significant proportion (approximately 20%) of women with pelvic masses have malignant disease and the proportion becomes even higher in postmenopausal women. Thus, the majority of women who undergo surgery for a suspected ovarian cancer do not, in fact, have cancer and if there were a reliable way to diagnose ovarian cancer, the savings in morbidity and mortality would be substantial, as would the associated financial costs. Approximately 22,000 women are diagnosed with ovarian cancer in the U.S. annually, and approximately 16,000 die of the disease. Since 5 or more women undergo evaluation for possible ovarian cancer for every woman who is eventually diagnosed with the disease, more than 100,000 women each year would benefit from a test which could reliably diagnose ovarian cancer.

### BRIEF SUMMARY OF THE INVENTION

The subject invention concerns methods for detecting, diagnosing, and monitoring therapy of ovarian cancer in a patient. A method of the invention comprises qualitatively or quantitatively analyzing a biological sample from a female animal for the presence or absence, or amount, of one or more proteins and/or nucleic acids associated with normal or cancerous cells of the ovaries.

The subject invention also concerns compositions that can be used to detect ovarian cancer marker proteins. In one embodiment, a composition of the invention comprises an array or panel of binding moities that can bind to a specific ovarian cancer marker protein. Examples of binding moities include antibodies, peptides, nucleic acids, aptamers, and marker protein ligands.

### BRIEF DESCRIPTION OF THE DRAWINGS

FIG. 1 shows the workflow process for analysis of plasma samples by 2D-DIGE.

FIG. 2 shows the workflow process samples.

FIGS. 3A and 3B show a comparison of Phase 2 (having 50 cases) and Phase 3 (having 52 cases) results leading to the selection of five candidate proteins.

FIG. 4 is a representative gel image showing the 5 candidate protein spots that show statistically significant difference (p≦0.05) between Disease and Normal. Protein E was detected only in 47% of cases.

FIGS. 5A and 5B show a comparison of candidate protein A in “normal” and “disease” samples.

FIGS. 6A and 6B show a comparison of candidate protein B in “normal” and “disease” samples.

FIGS. 7A and 7B show a comparison of candidate protein C in “normal” and “disease” samples.

FIGS. 8A and 8B show a comparison of candidate protein D in “normal” and “disease” samples.

FIGS. 9A and 9B show a comparison of candidate protein E in “normal” and “disease” samples.

FIGS. 10A and 10B show a comparison of candidate protein F in “normal” and “disease” samples.

### DETAILED DESCRIPTION OF THE INVENTION

The present invention concerns methods of using identified proteins that are present at different levels in a biological sample of a female with ovarian cancer as compared with a healthy female. Ovarian cancer cells produce proteins that are different from those produced by normal ovarian cells. Some of these proteins end up in the circulation and can be measured in the blood of women with ovarian cancer. Included in the invention are proteins in the blood of women with ovarian cancer compared with women who are healthy. A group of proteins have been identified that differ between the two groups. Measurement of these proteins can be used to diagnose ovarian cancer, to screen for ovarian cancer, and/or to monitor treatments of ovarian cancer in patients. A clinical test that can reliably detect and/or diagnose ovarian cancer and provide a means for screening for disease and monitoring therapy being given to a patient is provided.

In one embodiment, a method of the invention comprises qualitatively or quantitatively analyzing or measuring a biological sample from a female animal for the presence or absence, or amount or concentration, of one or more proteins and/or nucleic acids associated with the presence of ovarian cancer in a subject. The analysis or measurement of the proteins and/or nucleic acid can be correlated with the status of ovarian cancer in the subject, e.g., no cancer present, cancer present, stage of cancer present, risk of cancer, effectiveness of treatment, etc. Proteins (and nucleic acids that encode all or a fragment of the proteins) that can be screened for and analyzed in a sample include, but are not limited to, ceruloplasmin, keratin 10 (cytokeratin 10), haptoglobin, GTP binding protein, leucine-rich alpha-2-glycoprotein, alpha-1-acid glycoprotein, HP protein (histidine), alpha-1-anti proteinase (Clade A), immunoglobulin heavy chain, alpha-1-microglobin/bikunin precursor, poly ubiquitin C, human cystatin A, dermicidin precursor, AIDD protein, hemoglobin delta chain, hemoglobin alpha chain, hemopexin, human IgG1, serine/cysteine protease inhibitor, clusterin, ficolin, amyloid P component, and any combination thereof. All forms of a protein of the invention, e.g., variants and fragments, such as splice variants or allelic variants, glycosylation variants, phosphorylation variants, proteolytic cleavage variants, etc., are contemplated within the scope of the invention. In a specific embodiment, the methods comprise screening for and analyzing a sample for the proteins haptoglobin and α-1 microglobulin/bikunin precursor. Optionally, the level of a marker protein or nucleic acid in a sample can be compared to a control reference standard of the same protein or nucleic acid. The methods of the invention can be used in conjunction with other assays and methodologies for screening for ovarian cancer, e.g., tissue biopsy.

The proteins can be detected and analyzed using any suitable method. In one embodiment, proteins are analyzed and detected using an antibody-based assay. Antibodies specifically reactive with a marker protein, or derivatives, such as enzyme conjugates or labeled derivatives, can be used to detect the marker protein in various biological samples, for example they may be used in any known immunoassays which rely on the binding interaction between an antigenic determinant of a protein and the antibodies. Examples of such assays are radioimmunoassay (RIA), enzyme immunoassay (e.g., ELISA), Western blotting, immunofluorescence, immunoprecipitation, latex agglutination, hemagglutination, and histochemical tests. In a further embodiment, a protein can be detected and analyzed using chromatographic techniques (e.g., HPLC, gel electrophoresis) and/or mass spectrometry (e.g., MS/MS, LC-MS/MS, GC-MS, MALDI-T of MS, SELDI-MS). In another embodiment, proteins and nucleic acids can be analyzed using standard sequencing methods known in the art.

In one embodiment, marker proteins for ovarian cancer can be identified, analyzed, and quantified using quantitative mass spectrometric multiple reaction monitoring (MRM) methodologies (see, for example, Anderson and Hunter (2006); Kuhn et al. (2004)). Specific tryptic peptides can be selected as stoichiometric representatives of the proteins from which they are cleaved and quantitated against a stable isotope-labeled peptide as an internal standard to provide a measure of the concentration of the protein. The MRM methods can be coupled with procedures for enrichment of proteins such as immunodepletion and size exclusion chromatography (Liao et al. (2004)) and peptide enrichment using antibody capture (SISCAPA) (Anderson et al. (2004)).

An antibody specific for the marker protein can be labeled with a detectable substance and localized in biological samples based upon the presence of the detectable substance. Examples of detectable substances include, but are not limited to, the following radioisotopes (e.g., ³H, ¹⁴C, ³⁵S, ¹²⁵I, ¹³¹I) fluorescent labels (e.g., FITC, rhodamine, lanthanide phosphors), quantum dots (e.g., Qdot nanocrystals are nanometer size atom clusters containing atoms of a semiconductor material (e.g., cadmium mixed with selenium or tellurium) which has been coated with an additional semiconductor shell (e.g., zinc oxide)), luminescent labels such as luminol; enzymatic labels (e.g., horseradish peroxidase, beta-galactosidase, luciferase, alkaline phosphatase, acetylcholinestease), biotinyl groups (which can be detected by marked avidin, e.g., streptavidin containing a fluorescent marker or enzymatic activity that can be detected by optical or calorimetric methods), predetermined polypeptide epitopes recognized by a secondary reporter (e.g., leucine zipper pair sequences, binding sites for secondary antibodies, metal binding domains, epitope tags). Indirect methods may also be employed in which the primary antigen-antibody reaction is amplified by the introduction of a second antibody, having specificity for the antibody reactive against the marker protein. By way of example, if the antibody having specificity against the marker protein is a rabbit IgG antibody, the second antibody may be goat anti-rabbit gamma-globulin labeled with a detectable substance as described herein.

Methods for conjugating or labeling the antibodies discussed above may be readily accomplished by one of ordinary skill in the art. (See, for example, Imman (1974) and Wilchek and Bayer (1988), regarding methods for conjugating or labeling the antibodies with an enzyme or ligand binding partner).

Time-resolved fluorometry may be used to detect a signal. For example, the method described in Christopoulos and Diamandis (1992) may be used with a conventional time-resolved fluorometer.

Therefore, in accordance with one embodiment of the invention, a method is provided wherein an antibody to a marker protein is labeled with an enzyme, a substrate for the enzyme is added wherein the substrate is selected so that the substrate, or a reaction product of the enzyme and substrate, forms fluorescent complexes with a lanthanide metal. A lanthanide metal is added and the marker protein is quantitated in the sample by measuring fluorescence of the fluorescent complexes. Antibodies specific for the marker protein may be directly or indirectly labeled with an enzyme. Enzymes are selected based on the ability of a substrate of the enzyme, or a reaction product of the enzyme and substrate, to complex with lanthanide metals such as europium and terbium. Examples of suitable enzymes include alkaline phosphatase and beta-galactosidase. Preferably, the enzyme is alkaline phosphatase. Antibodies may also be indirectly labeled with an enzyme. For example, the antibodies may be conjugated to one partner of a ligand binding pair, and the enzyme may be coupled to the other partner of the ligand binding pair. Representative examples include avidin-biotin, and riboflavin-riboflavin binding protein. Preferably the antibodies are biotinylated, and the enzyme is coupled to streptavidin.

In an embodiment of the method, antibody bound to a marker protein in a sample is detected by adding a substrate for the enzyme. The substrate is selected so that in the presence of a lanthanide metal (e.g., europium, terbium, samarium, and dysprosium, preferably europium and terbium), the substrate or a reaction product of the enzyme and substrate, forms a fluorescent complex with the lanthanide metal. Examples of enzymes and substrates for enzymes that provide such fluorescent complexes are described in U.S. Pat. No. 5,312,922 to Diamandis. By way of example, when the antibody is directly or indirectly labeled with alkaline phosphatase, the substrate employed in the method may be 4-methylumbeliferyl phosphate, or 5-fluorosalicyl phosphate. The fluorescence intensity of the complexes can be measured, for example, using a time-resolved fluorometer, e.g., a CyberFluor 615 Immunoanalyzer (Nordion International, Kanata Ontario).

The sample, the antibody specific for the marker protein, or the marker protein, may be immobilized on a carrier. Examples of suitable carriers are agarose, cellulose, dextran, Sephadex, Sepharose, liposomes, carboxymethyl cellulose polystyrene, filter paper, ion-exchange resin, plastic film, plastic tube, glass beads, polyamine-methyl vinyl ether-maleic acid copolymer, amino acid copolymer, ethylene-maleic acid copolymer, nylon, silk, etc. The carrier may be in the shape of, for example, a tube, test plate, well, beads, disc, sphere, etc. The immobilized antibody may be prepared by reacting the material with a suitable insoluble carrier using known chemical or physical methods, for example, cyanogen bromide coupling.

In accordance with one embodiment, the present invention provides a mode for determining a marker protein in a sample by measuring the marker protein by immunoassay. It will be evident to a skilled artisan that a variety of immunoassay methods can be used to measure the marker protein. In general, an immunoassay method may be competitive or noncompetitive. Competitive methods typically employ an immobilized or immobilizable antibody to the marker protein and a labeled form of the marker protein. The marker protein in the sample and the labeled marker protein compete for binding to the antibody. After separation of the resulting labeled marker protein that has become bound to the antibody (bound fraction) from that which has remained unbound (unbound fraction), the amount of the label in either bound or unbound fraction is measured and may be correlated with the amount of the marker protein in the biological sample in any conventional manner, e.g., by comparison to a standard curve.

A noncompetitive method can also be used for the determination of a marker protein, with the most common method being the “sandwich” method. In this assay, two antibodies, both of which bind to the marker protein, are employed. One of the antibodies is directly or indirectly labeled (also referred to as the “detection antibody”) and the other antibody is immobilized or immobilizable (also referred to as the “capture antibody”). The capture and detection antibodies can be contacted simultaneously or sequentially with the biological sample. Sequential methods can be accomplished by incubating the capture antibody with the sample, and adding the detection antibody at a predetermined time thereafter (sometimes referred to as the “forward” method); or the detection antibody can be incubated with the sample first and then the capture antibody added (sometimes referred to as the “reverse” method). After the necessary incubation(s) have occurred, to complete the assay, the capture antibody is separated from the liquid test mixture, and the label is measured in at least a portion of the separated capture antibody phase or the remainder of the liquid test mixture. Generally, it is measured in the capture antibody phase since it comprises the marker protein bound by (“sandwiched” between) the capture and detection antibodies.

In a typical two-site immunometric assay for a marker protein, one or both of the capture and detection antibodies are polyclonal antibodies. The label used in the detection antibody can be selected from any of those known conventionally in the art. As with other embodiments of the protein detection assay, the label can be an enzyme or a chemiluminescent moiety, for example, or a radioactive isotope, a fluorophore, a quantum dot, a detectable ligand (e.g., detectable by a secondary binding by a labeled binding partner for the ligand), and the like. Preferably, the antibody is labeled with an enzyme that is detected by adding a substrate that is selected so that a reaction product of the enzyme and substrate forms fluorescent complexes. The capture antibody is selected so that it provides a mode for being separated from the remainder of the test mixture. Accordingly, the capture antibody can be introduced to the assay in an already immobilized or insoluble form, or can be in an immobilizable form, that is, a form which enables immobilization to be accomplished subsequent to introduction of the capture antibody to the assay. An immobilized capture antibody can comprise an antibody covalently or noncovalently attached to a solid phase such as a magnetic particle, a latex particle, a microtiter multi-well plate, a bead, a cuvette, or other reaction vessel. An example of an immobilizable capture antibody is an antibody that has been chemically modified with a ligand moiety, e.g., a hapten, biotin, or the like, and that can be subsequently immobilized by contact with an immobilized form of a binding partner for the ligand, e.g., an antibody, avidin, or the like. In one embodiment, the capture antibody can be immobilized using a species specific antibody for the capture antibody that is bound to the solid phase.

A particular sandwich immunoassay method of the invention employs two antibodies reactive against a marker protein, a second antibody having specificity against an antibody reactive against the marker protein labeled with an enzymatic label, and a fluorogenic substrate for the enzyme. In one embodiment, the enzyme is alkaline phosphatase (ALP) and the substrate is 5-fluorosalicyl phosphate. ALP cleaves phosphate out of the fluorogenic substrate, 5-fluorosalicyl phosphate, to produce 5-fluorosalicylic acid (FSA). 5-Fluorosalicylic acid can then form a highly fluorescent ternary complex of the form FSA-Tb(3+)-EDTA, which can be quantified by measuring the Tb³⁺ fluorescence in a time-resolved mode. Fluorescence intensity is typically measured using a time-resolved fluorometry as described herein.

The above-described immunoassay methods and formats are intended to be exemplary and are not limiting since, in general, it will be understood that any immunoassay method or format can be used in the present invention.

Expression of a protein associated with ovarian cancer in a subject can be elevated as compared to expression of that same protein in a normal subject that does not have ovarian cancer. Similarly, expression of a protein associated with ovarian cancer in a subject can decrease as compared to expression of that same protein in a normal subject that does not have ovarian cancer. The increase or decrease in expression can be viewed as a ratio of protein expression levels in normal subjects versus subjects having ovarian cancer. As used herein, a ratio of protein expression level having a positive value represents that the particular protein is found at elevated levels in a subject having ovarian cancer as compared to a subject that does not have ovarian cancer. A ratio of protein expression level having a negative value represents that the particular protein is found at lower levels in a subject having ovarian cancer as compared to a subject that does not have ovarian cancer. In one embodiment, where the level of expression of a particular protein is elevated in a subject having ovarian cancer, the ratio for the particular protein can be about 1.3 or greater, or about 1.5 or greater, or about 2.0 or greater, or about 4.0 or greater. In one embodiment, where the level of expression of a particular protein is decreased in a subject having ovarian cancer, the ratio for the particular protein can be about −1.3 or less, −1.5 or less, or −2.0 or less.

The presence or amount of a protein associated with a subject having ovarian cancer can be compared to a reference control for that protein to determine if the level of protein corresponds to the level of the protein typically found in a normal subject or to the level of the protein typically found in a subject with ovarian cancer. For example, if the level of a marker protein in a patient having ovarian cancer is about twice the level of the same protein in a patient that does not have ovarian cancer, then a biological sample to be assayed can be analyzed for the presence and level of the marker protein and compared against a reference control level of that protein in a normal subject and/or in a subject having ovarian cancer.

Nucleic acids include naturally occurring nucleic acids, oligonucleotides, antisense oligonucleotides, and synthetic oligonucleotides that hybridize to the nucleic acid encoding an ovarian cancer marker protein. The present invention contemplates the use of nucleic acid sequences corresponding to the coding sequence of the marker protein and to the complementary sequence thereof, as well as sequences complementary to the transcript sequences occurring further upstream or downstream from the coding sequence (e.g., sequences contained in, or extending into, the 5′ and 3′ untranslated regions) for use as agents for detecting the expression of the marker protein in biological samples of ovarian cancer patients, or those at risk of ovarian cancer.

The preferred oligonucleotides for detecting the presence of nucleic acid encoding a marker protein in biological samples are those that are complementary to at least part of an RNA or DNA sequence encoding the marker protein. Oligonucleotides may be oligoribonucleotides or oligodeoxyribonucleotides. In addition, oligonucleotides may be natural oligomers composed of the biologically significant nucleotides, i.e., A (adenine), dA (deoxyadenine), G (guanine), dG (deoxyguanine), C (cytosine), dC (deoxycytosine), T (thymine) and U (uracil), or modified oligonucleotide species, substituting, for example, a methyl group or a sulfur atom for a phosphate oxygen in the inter-nucleotide phosohodiester linkage. Additionally, these nucleotides themselves, and/or the ribose moieties may be modified.

The oligonucleotides may be synthesized chemically, using any of the known chemical oligonucleotide synthesis methods well described in the art. For example, the oligonucleotides can be prepared by using any of the commercially available, automated nucleic acid synthesizers. Alternatively, the oligonucleotides may be created by standard recombinant DNA techniques, for example, inducing transcription of the noncoding strand. The DNA sequence encoding the marker protein may be inverted in a recombinant DNA system, e.g., inserted in reverse orientation downstream of a suitable promoter, such that the noncoding strand now is transcribed.

Although any length oligonucleotide may be utilized to hybridize to a nucleic acid encoding a marker protein, oligonucleotides typically within the range of 8-100 nucleotides are generally used. In one embodiment, oligonucleotides for use in detecting a marker protein can be within the range of 15-50 nucleotides.

An oligonucleotide selected for hybridizing to a nucleic acid molecule, whether synthesized chemically or by recombinant DNA technology, can be isolated and purified using standard techniques and then optionally labeled (e.g., with ³⁵S or ³²P) using standard labeling protocols.

The present invention also contemplates the use of oligonucleotide pairs in polymerase chain reactions (PCR) to detect a nucleic acid encoding a marker protein of the invention in biological samples. The oligonucleotide pairs can include a forward primer and a reverse primer.

The presence of a nucleic acid encoding a marker protein of the invention in a sample from a patient may be determined by nucleic acid hybridization, such as but not limited to Northern blot analysis, dot blotting, Southern blot analysis, fluorescence in situ hybridization (FISH), and PCR. Chromatography, such as HPLC, and other known assays may also be used to determine messenger RNA levels in a sample.

In one aspect, the present invention contemplates the use of nucleic acids as agents for detecting ovarian cancer marker proteins in biological samples of patients, wherein the nucleic acids are labeled. The nucleic agents may be labeled with a radioactive label, a fluorescent label, a quantum dot, an enzyme, a chemiluminescent tag, a colorimetric tag or other labels or tags that are discussed above or that are known in the art.

In another aspect, the present invention contemplates the use of Northern blot analysis to detect the presence of ovarian cancer marker protein mRNA in a biological sample. The first step of the analysis involves separating a sample containing nucleic acid by gel electrophoresis. The dispersed nucleic acids are then transferred to a nitrocellulose filter or another filter. Subsequently, the filter is contacted with labeled oligonucleotide under suitable hybridizing conditions, e.g., 50% formamide, 5×SSPE, 2×Denhardt's solution, 0.1% SDS at 42° C., as described in Molecular Cloning: A Laboratory Manual, Maniatis et al. (1982, CSH Laboratory). Other useful procedures known in the art include solution hybridization, dot and slot RNA hybridization, and probe based microarrays. Measuring the radioactivity of hybridized fragments, using standard procedures known in the art quantitates the amount of a particular nucleic acid present in the biological fluid of a patient.

Dot blotting involves applying samples that may contain a nucleic acid of interest to a membrane. The nucleic acid can be denatured before or after application to the membrane. The membrane is incubated with a labeled probe. Dot blot procedures are well known to the skilled artisan and are described more fully in U.S. Pat. Nos. 4,582,789 and 4,617,261, the disclosures of which are incorporated herein by reference.

Polymerase chain reaction (PCR) is a process for amplifying one or more specific nucleic acid sequences present in a nucleic acid sample using primers and agents for polymerization and then detecting the amplified sequence. The extension product of one primer when hybridized to the other becomes a template for the production of the desired specific nucleic acid sequence, and vice versa, and the process is repeated as often as is necessary to produce the desired amount of the sequence. PCR is routinely used to detect the presence of a desired sequence (U.S. Pat. No. 4,683,195).

A specific example of PCR that is routinely performed by the skilled artisan to detect desired sequences is reverse transcription PCR (RT-PCR; Saiki et al. (1985) and Scharf et al. (1986)). RT-PCR involves isolating total RNA from biological fluid, denaturing the RNA in the presence of primers that recognize the desired nucleic acid sequence, using the primers to generate a cDNA copy of the RNA by reverse transcription, amplifying the cDNA by PCR using specific primers, and detecting the amplified cDNA by electrophoresis or other methods known to the skilled artisan. The amount of a target nucleic acid sequence in a sample can be quantitated using standard PCR methods.

In one embodiment, a method of the present invention is used to detect, diagnose, and/or monitor therapy of early stage ovarian cancer. Proteins that can be assayed for in the method for early stage ovarian cancer include, but are not limited to, hemopexin, human IgG1, haptoglobin, serine/cysteine protease inhibitor, clusterin, ficolin, alpha-1-microglobin/bikunin precursor, and amyloid P component, or any combination thereof. In one embodiment, the protein assayed for in a method for early stage ovarian cancer is hemopexin and/or haptoglobulin.

In another embodiment, a method of the present invention is used to detect, diagnose, and/or monitor therapy of late stage ovarian cancer. Proteins that can be assayed for in the method for late stage ovarian cancer include, but are not limited to, ceruloplasmin, keratin 10 (cytokeratin 10), haptoglobin, GTP binding protein, leucine-rich alpha-2-glycoprotein, alpha-1-acid glycoprotein, HP protein (histidine), alpha-1-anti proteinase (Clade A), immunoglobulin heavy chain, alpha-1-microglobin/bikunin precursor, poly ubiquitin C, human cystatin A, dermicidin precursor, AIDD protein, and hemoglobin delta chain, or any combination thereof.

The methods of the invention can be used to establish a prognosis and/or to design, determine, and/or monitor therapeutic treatments on a subject having ovarian cancer. For example, the presence or levels of ovarian cancer marker proteins or nucleic acids in a subject can be monitored prior to treatment, such as chemotherapy, radiation, and/or surgery, and/or monitored during and after a treatment regimen is completed. The methods of the invention can also be used to monitor for remission or relapse of a subject.

The results obtained from using a method of the invention can be recorded on a tangible medium and/or reported to the subject. The results obtained can also be used by a clinician to manage therapeutic treatments and protocols that the patient may receive.

In one embodiment, methods of the invention further comprise identifying one or more proteins that are differentially expressed in subjects having ovarian cancer compared to normal subjects that do not have ovarian cancer. The identification step can be directed to identifying one or more proteins that are differentially expressed in subjects having early stage ovarian cancer, or to identifying one or more proteins that are differentially expressed in subjects having late stage ovarian cancer. Thus, the methods can be used to provide a diagnosis directed to early stage or late stage ovarian cancer so that treatment appropriate for the specific stage of the disease can be instituted.

In some embodiments, the subject exhibits no symptoms of ovarian cancer at the time a method of the invention is carried out. In other embodiments, the subjects exhibit one or more symptoms of ovarian cancer at the time a method of the invention is carried out. For example, with respect to ovarian cancer, the one or more symptoms may include pelvic pain, abnormal vaginal bleeding, abdominal swelling or bloating, persistent back pain, persistent stomach upset, change in bowel or bladder pattern (such as constipation, diarrhea, blood in the stools, gas, thinner stools, frequency or urgency of urination, constipation), pain during intercourse, unintentional weight loss of ten or more pounds, vulva or vaginal abnormality (such as blister, change in skin color, or discharge), change in the breast (such as a lump, soreness, nipple discharge, dimpling, redness, or swelling), and/or fatigue.

The subject invention also concerns compositions that can be used to detect ovarian cancer marker proteins that are differentially expressed in subjects having ovarian cancer as compared to subjects that do not have ovarian cancer. In one embodiment, a composition of the invention comprises one or more isolated ovarian cancer protein markers, or nucleic acid encoding them, which can optionally be provided as part of an array, panel, container, etc. In another embodiment, a composition of the invention comprises a panel or array of antibodies, or antigen binding fragments thereof, which can specifically bind to an ovarian cancer marker protein. The antibodies can be monoclonal or polyclonal antibodies. Antigen binding fragments include, but are not limited to, F(ab′)2, Fab′,Fab, and Fv. In another embodiment, a composition of the invention comprises a panel or array of peptides or nucleic acids (e.g., aptamers) that can specifically bind to an ovarian cancer marker protein. In another embodiment, a composition of the invention comprises a panel or array of ligands that can bind specifically to an ovarian cancer marker protein. For example, if the ovarian cancer marker protein is a receptor protein, the ligand can be the natural biological ligand that binds to the receptor protein or a synthetic ligand that has been designed to bind to the receptor protein. Binding moieties of the invention, such as antibodies, peptides, and aptamers, that can bind to an ovarian cancer marker protein of the invention can be prepared using standard methods and materials in the art, or may be commercially available. Compositions of the invention can be provided on a solid phase support, such as plastic or nitrocellulose. Compositions of the invention can also include a reference control for one or more ovarian cancer marker proteins wherein a predetermined amount of the protein is provided. For example, a reference control protein can be provided such that if the protein is present in a sample, the level or amount of the protein present can be compared to the level or amount of the same protein typically found in a subject having ovarian cancer and/or a subject that does not have ovarian cancer.

In one aspect, the present invention includes kits comprising the required elements for diagnosing or monitoring cancer. In one embodiment, the kits comprise a container for collecting a biological sample from a patient and an agent for detecting and/or quantifying the presence of an ovarian cancer marker protein of the invention or nucleic acid encoding it. The components of the kits can be packaged either in aqueous medium or in lyophilized form.

The methods of the invention can be carried out using a diagnostic kit for qualitatively or quantitatively detecting an ovarian cancer marker protein of the invention in a sample such as blood or urine. By way of example, the kit can contain binding agents (e.g., antibodies) specific for an ovarian cancer marker protein of the invention, antibodies against the antibodies labeled with an enzyme; and a substrate for the enzyme. The kit can also contain a solid support such as microtiter multi-well plates, standards, assay diluent, wash buffer, adhesive plate covers, and/or instructions for carrying out a method of the invention using the kit. In one embodiment, the kit includes one or more protease inhibitors (e.g., a protease inhibitor cocktail) to be applied to the biological sample to be assayed (such as blood or urine).

Kits for diagnosing or monitoring ovarian cancer containing one or more agents that detect a marker protein, such as but not limited to antibodies, or fragments thereof, or other binding moiety, can be prepared. The agent(s) can be packaged with a container for collecting the biological fluid from a patient. When the antibodies or binding moiety are used in the kits in the form of conjugates in which a label is attached, such as a radioactive metal ion or a moiety, the components of such conjugates can be supplied either in fully conjugated form, in the form of intermediates or as separate moieties to be conjugated by the user of the kit.

Kits containing one or more agents that detect nucleic acid encoding an ovarian cancer marker protein, such as but not limited to the full length nucleic acid, oligonucleotides, and pairs of primers can also be prepared. The agent(s) can be packaged with a container for collecting biological samples from a patient. The nucleic acid can be in the labeled form or to be labeled form.

Other components of the kit may include but are not limited to, means for collecting biological samples, means for labeling the detecting agent (binding agent), membranes for immobilizing the marker protein or nucleic acid in the biological sample, means for applying the biological sample to a membrane, means for binding the agent to the marker protein or nucleic acid in the biological sample of a subject, a second antibody, a means for isolating total RNA from a biological fluid of a subject, means for performing gel electrophoresis, means for generating cDNA from isolated total RNA, means for performing hybridization assays, and means for performing PCR, etc.

As used herein, the term “ELISA” includes an enzyme-linked immunoabsorbent assay that employs an antibody or antigen bound to a solid phase and an enzyme-antigen or enzyme-antibody conjugate to detect and quantify the amount of an antigen or antibody present in a sample. A description of the ELISA technique is found in Sites et al. (1982) and in U.S. Pat. Nos. 3,654,090; 3,850,752; and 4,016,043, the disclosures of which are herein incorporated by reference. ELISA is an assay that can be used to quantitate the amount of antigen, proteins, or other molecules of interest in a sample. In particular, ELISA can be carried out by attaching on a solid support (e.g., polyvinylchloride) an antibody specific for an antigen or protein of interest. Cell extract or other sample of interest such as urine can be added for formation of an antibody-antigen complex, and the extra, unbound sample is washed away. An enzyme-linked antibody, specific for a different site on the antigen is added. The support is washed to remove the unbound enzyme-linked second antibody. The enzyme-linked antibody can include, but is not limited to, alkaline phosphatase. The enzyme on the second antibody can convert an added colorless substrate into a colored product or can convert a non-fluorescent substrate into a fluorescent product. The ELISA-based assay method provided herein can be conducted in a single chamber or on an array of chambers and can be adapted for automated processes.

In these exemplary embodiments, the antibodies can be labeled with pairs of FRET dyes, bioluminescence resonance energy transfer (BRET) protein, fluorescent dye-quencher dye combinations, beta gal complementation assays protein fragments. The antibodies may participate in FRET, BRET, fluorescence quenching or beta-gal complementation to generate fluorescence, colorimetric or enhanced chemiluminescence (ECL) signals, for example.

These methods are routinely employed in the detection of antigen-specific antibody responses, and are well described in general immunology text books such as Roitt et al. (1998) and Janeway and Travers (1994), the contents of which are herein incorporated by reference.

The methods of the present invention can be used with female humans and other animals. The other animals contemplated within the scope of the invention include domesticated, agricultural, or zoo- or circus-maintained animals. Domesticated animals include, for example, dogs, cats, rabbits, ferrets, guinea pigs, hamsters, pigs, monkeys or other primates, and gerbils. Agricultural animals include, for example, horses, mules, donkeys, burros, cattle, cows, pigs, sheep, and alligators. Zoo- or circus-maintained animals include, for example, lions, tigers, bears, camels, giraffes, hippopotamuses, and rhinoceroses.

Biological samples refer to a composition obtained from a human or animal. Biological samples within the scope of the invention include, but are not limited to, whole blood, blood plasma, serum, urine, tears, saliva, sputum, exhaled breath, nasal secretions, pharyngeal exudates, bronchoalveolar lavage, tracheal aspirations, interstitial fluid, lymph fluid, meningal fluid, amniotic fluid, glandular fluid, feces, perspiration, mucous, vaginal or urethral secretion, cerebrospinal fluid, and transdermal exudate. A biological sample also includes experimentally separated fractions of all of the preceding solutions or mixtures containing homogenized solid material, such as feces, tissues, and biopsy samples.

Samples and/or binding moieties may be arrayed on a solid support, or multiple supports can be utilized, for multiplex detection or analysis. “Arraying” refers to the act of organizing or arranging members of a library (e.g., an array of different samples), or other collection, into a logical or physical array. Thus, an “array” refers to a physical or logical arrangement of, e.g., biological samples. A physical array can be any “spatial format” or physically gridded format” in which physical manifestations of corresponding library members are arranged in an ordered manner, lending itself to combinatorial screening. For example, samples corresponding to individual or pooled members of a sample library can be arranged in a series of numbered rows and columns, e.g., on a multi-well plate. Similarly, binding moieties can be plated or otherwise deposited in microtitered, e.g., 96-well, 384-well, or-1536 well, plates (or trays). Optionally, binding moieties may be immobilized on the solid support.

Detection of cancer biomarkers, and other assays that are to be carried out on samples, can be carried out simultaneously or sequentially, and may be carried out in an automated fashion, in a high-throughput format.

As used herein, the terms solid “support”, “substrate”, and “surface” refer to a solid phase which is a porous or non-porous water insoluble material that can have any of a number of shapes, such as strip, rod, particle, beads, or multi-welled plate. In some embodiments, the support has a fixed organizational support matrix that preferably functions as an organization matrix, such as a microtiter tray. Solid support materials include, but are not limited to, cellulose, polysaccharide such as Sephadex, glass, polyacryloylmorpholide, silica, controlled pore glass (CPG), polystyrene, polystyrene/latex, polyethylene such as ultra high molecular weight polyethylene (UPE), polyamide, polyvinylidine fluoride (PVDF), polytetrafluoroethylene (PTFE; TEFLON), carboxyl modified teflon, nylon, nitrocellulose, and metals and alloys such as gold, platinum and palladium. The solid support can be biological, non-biological, organic, inorganic, or a combination of any of these, existing as particles, strands, precipitates, gels, sheets, pads, cards, strips, dipsticks, test strips, tubing, spheres, containers, capillaries, pads, slices, films, plates, slides, etc., depending upon the particular application. Preferably, the solid support is planar in shape, to facilitate contact with a biological sample such as urine, whole blood, plasma, serum, peritoneal fluid, or ascites fluid. Other suitable solid support materials will be readily apparent to those of skill in the art. The solid support can be a membrane, with or without a backing (e.g., polystyrene or polyester card backing), such as those available from Millipore Corp. (Bedford, Mass.), e.g., HI-FLOW Plus membrane cards. The surface of the solid support may contain reactive groups, such as carboxyl, amino, hydroxyl, thiol, or the like for the attachment of nucleic acids, proteins, etc. Surfaces on the solid support will sometimes, though not always, be composed of the same material as the support. Thus, the surface can be composed of any of a wide variety of materials, such as polymers, plastics, resins, polysaccharides, silica or silica-based materials, carbon, metals, inorganic glasses, membranes, or any of the aforementioned support materials (e.g., as a layer or coating).

As used herein, the terms “label” and “tag” refer to substances that may confer a detectable signal, and include, but are not limited to, enzymes such as alkaline phosphatase, glucose-6-phosphate dehydrogenase, and horseradish peroxidase, ribozyme, a substrate for a replicase such as QB replicase, promoters, dyes, quantum dots, fluorescers, such as fluorescein, isothiocynate, rhodamine compounds, phycoerythrin, phycocyanin, allophycocyanin, o-phthaldehyde, and fluorescamine, chemiluminescers such as isoluminol, sensitizers, coenzymes, enzyme substrates, radiolabels, particles such as latex or carbon particles, liposomes, cells, etc., which may be further labeled with a dye, catalyst or other detectable group.

As used in this specification, the singular forms “a”, “an”, and “the” include plural reference unless the context clearly dictates otherwise. Thus, for example, a reference to “an antibody” includes more than one such antibody. A reference to “a molecule” includes more than one such molecule, and so forth.

### Example 1

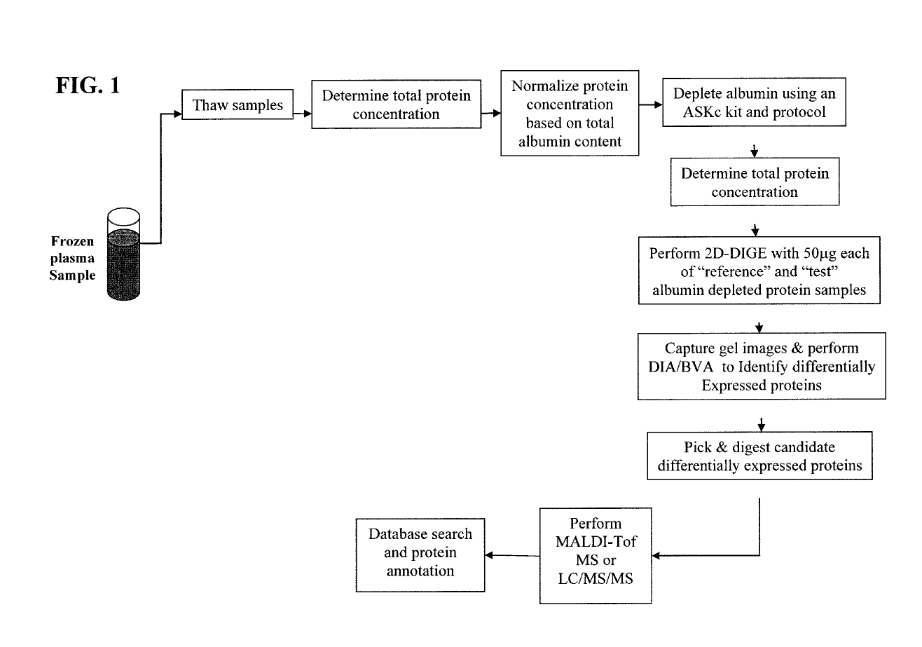

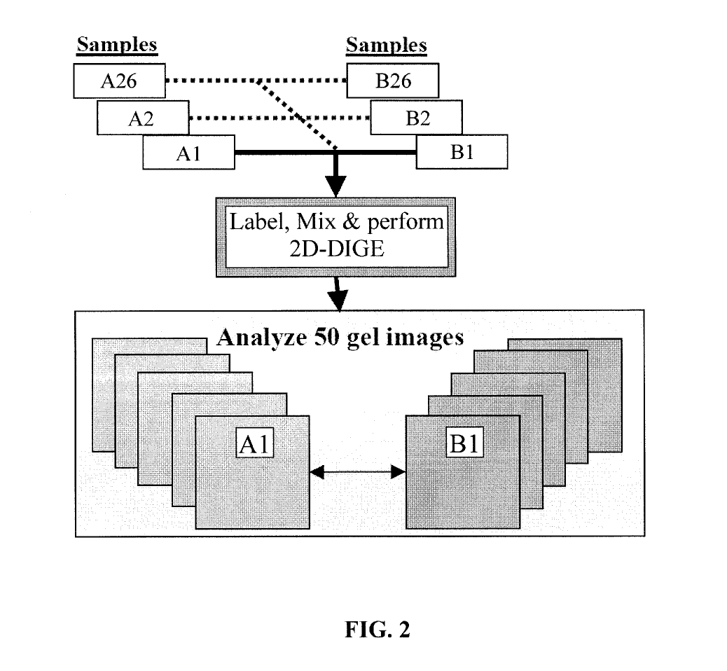

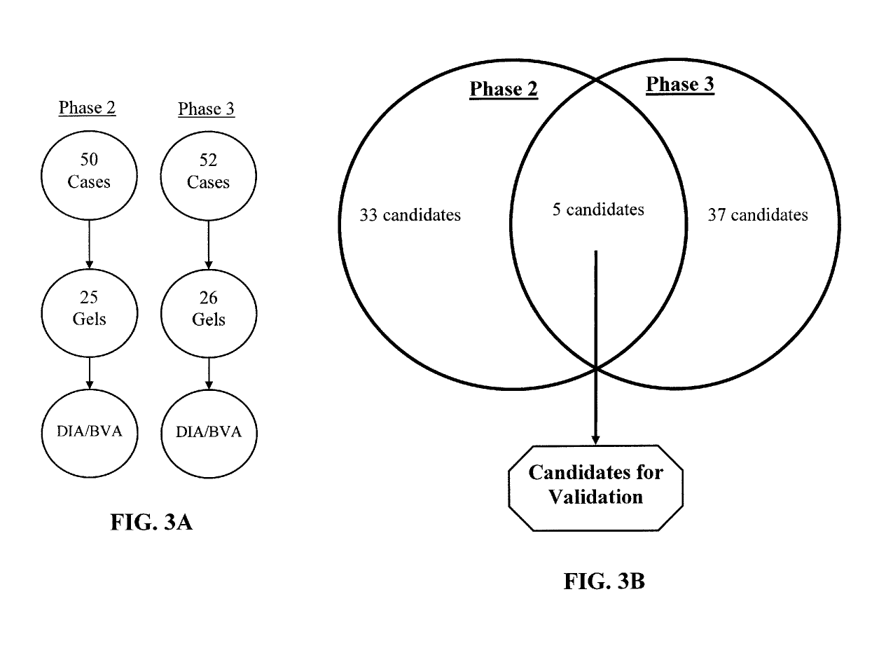

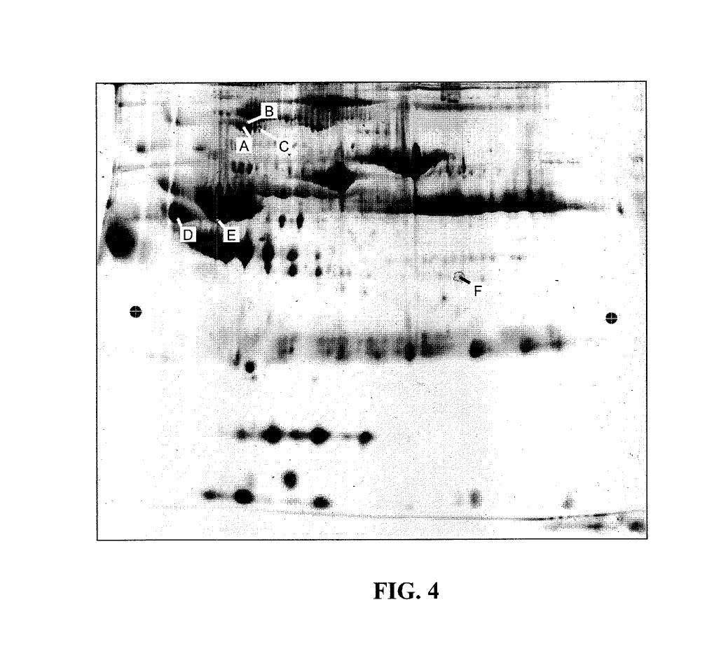

Plasma samples from test (n=26) and reference (n=26) donors were analyzed by 2D-DIGE after albumin depletion (FIGS. 2 and 3). Gels were scanned and the images generated were imported into DECYDER software (GE Healthcare) for statistical analysis. Identification of differentially expressed candidate spots were carried out with the False Discovery Mechanism (FDM) activated to increase overall confidence of spot selection. To be selected, candidate proteins must: 1) have been detected in at least 50% of the cases and 2) show statistically significant difference (p=≦0.05) in abundance between the “test” and “reference” samples. Candidate protein spots showing differential abundance (p=≦0.05) between “Test” (disease) and “Reference” (normal) were identified (FIG. 4), picked robotically, in-gel digested, and identity assigned by MALDI-TOF MS or LC MS/MS (see FIG. 1 and Table 1).

TABLE 1
Abundant proteins showing statistically significant difference (p ≦ 0.05)
in at least 50% of cases in the Phase 2 and Phase 3 studies*

| | Master No. | | | | Master No. | | | | | |
|---|---|---|---|---|---|---|---|---|---|---|
| Common | Batch 2 | Av Ratio | T-test | App | Batch 3 | Av. Ratio | T-test | App | NCBI ID | |
| Spots | (50 Samples) | Batch 2 | (p value) | (25 gels) | (52 Samples) | Batch 3 | (p value) | (26 gels) | Number | Protein ID |
| A | 562 | 1.93 | 0.006 | 17 | 372 | 1.47 | 0.042 | 18 | 1620909 | Ceruloplasmin |
| B | 566 | 1.95 | 0.006 | 20 | 376 | 1.64 | 0.014 | 16 | 1620909 | Ceruloplasmin |
| C | 596 | 1.99 | 0.059 | 19 | 391 | 1.46 | 0.03 | 16 | 71528 | Keratin 10 |
| D | 1458 | 2.46 | 0.018 | 13 | 1142 | 1.77 | 0.050 | 18 | 72059 | Leucine-Rich |
| | | | | | | | | | | alpha 2 |
| | | | | | | | | | | Glycoprotein |
| F | 1787 | −1.52 | 0.049 | 25 | 1439 | −1.64 | 0.014 | 25 | 10334547 | Immunoglobulin |
| | | | | | | | | | | Heavy Chain |

*Spot A and B proved to be the same protein, apparently differentially modified. Positive average ratios indicate that the candidate protein is more abundant in the plasma of donors with disease.

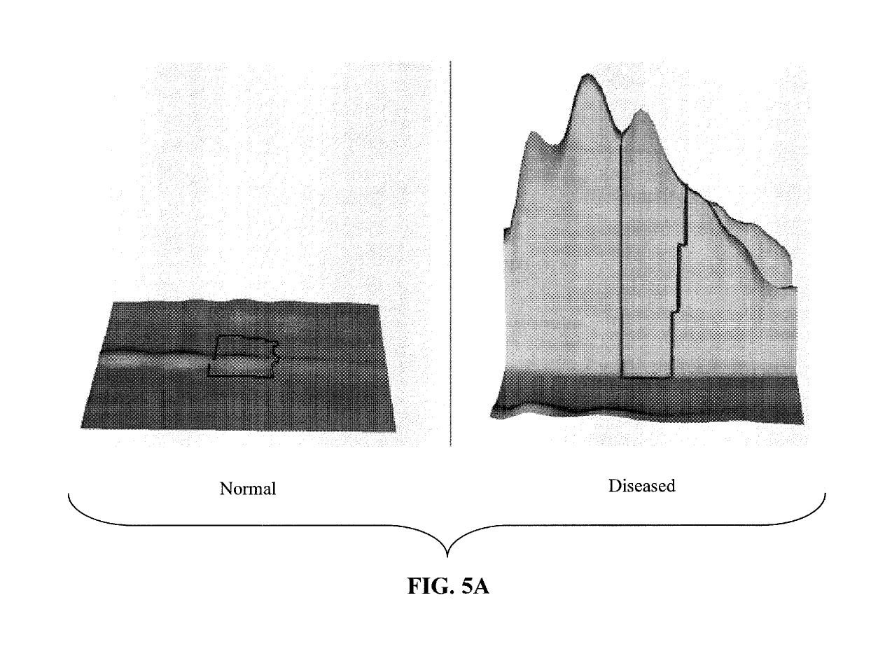

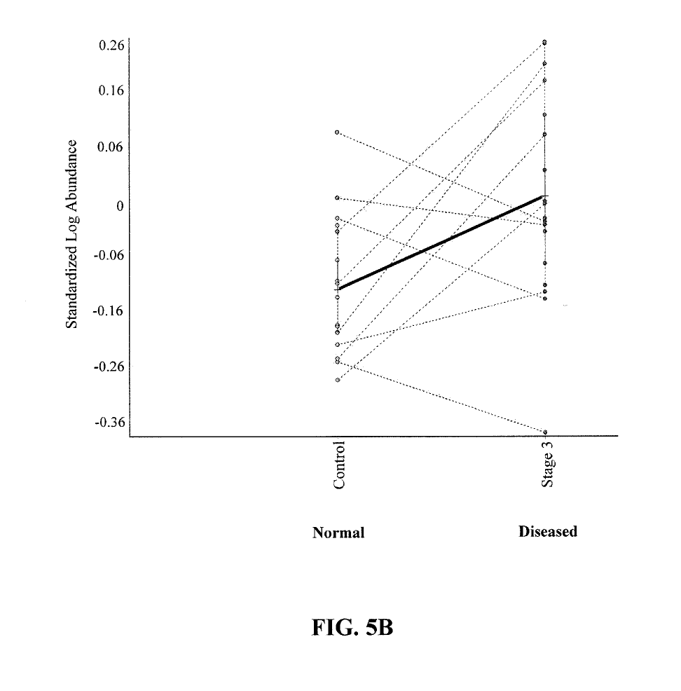

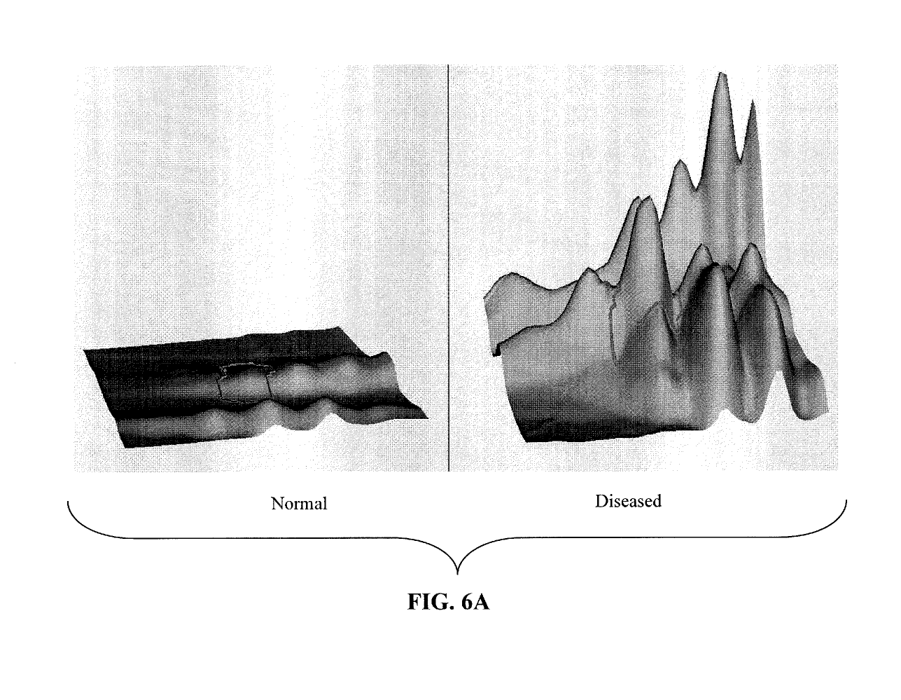

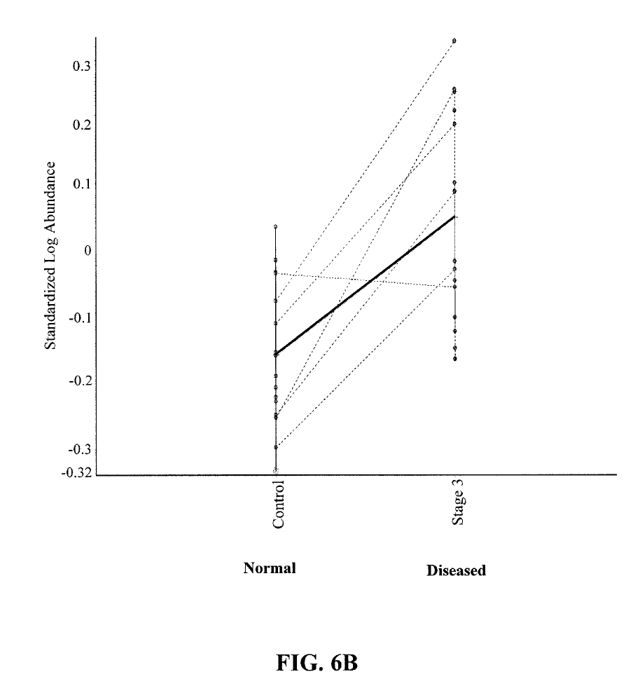

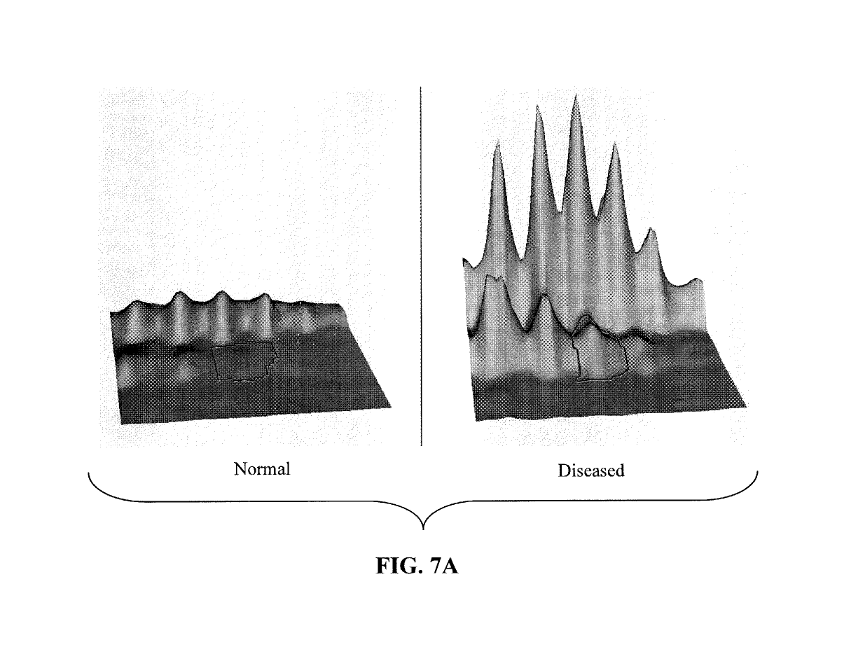

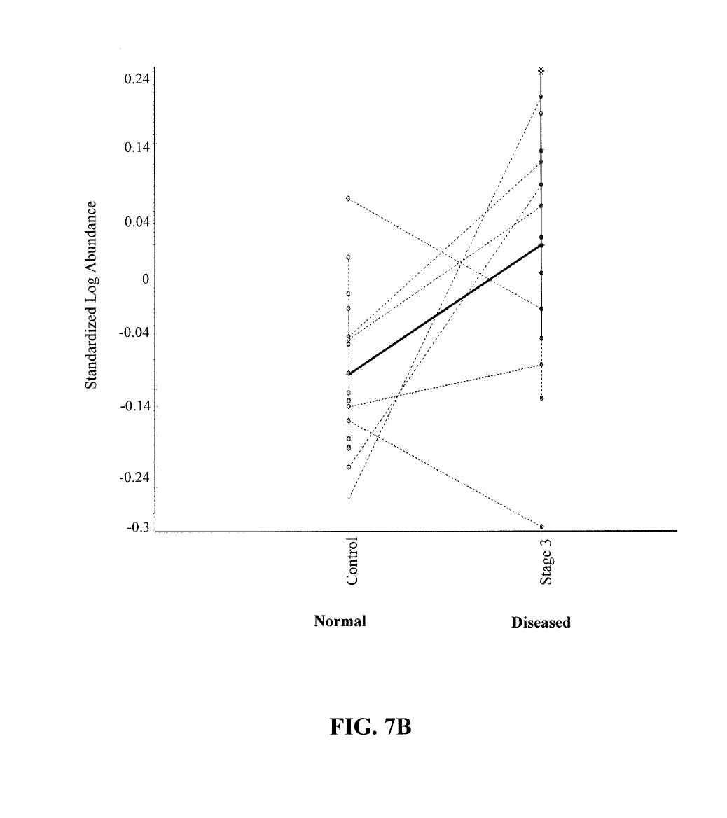

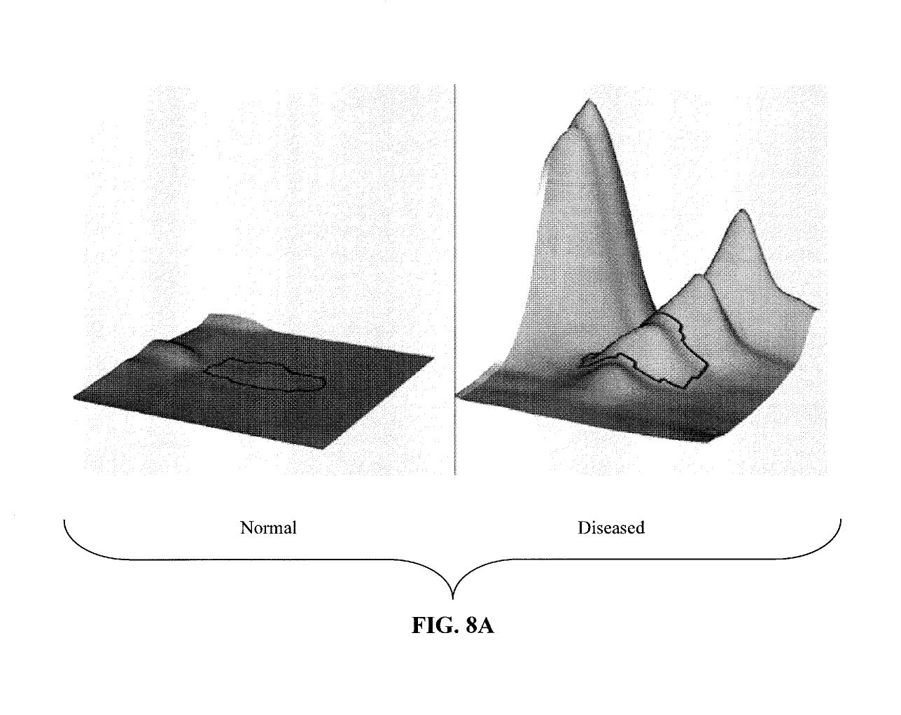

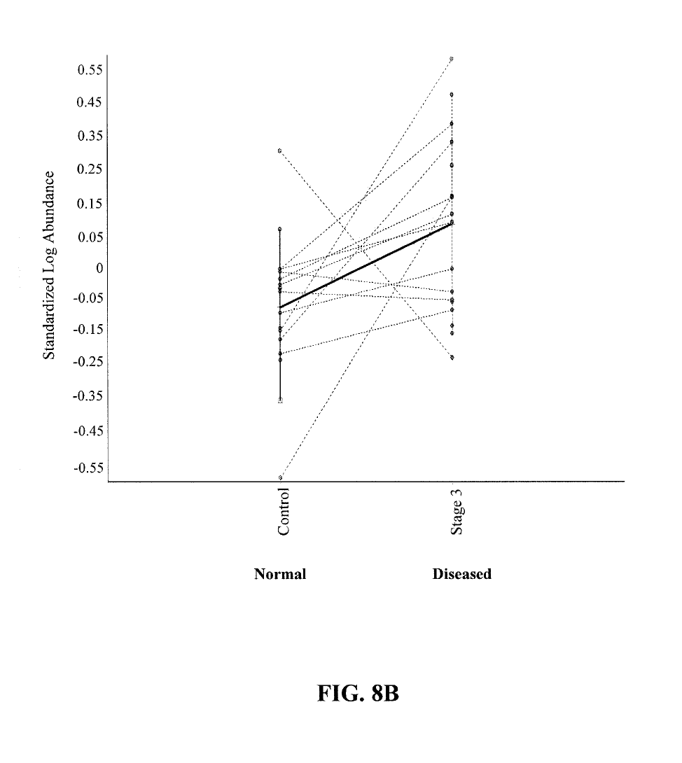

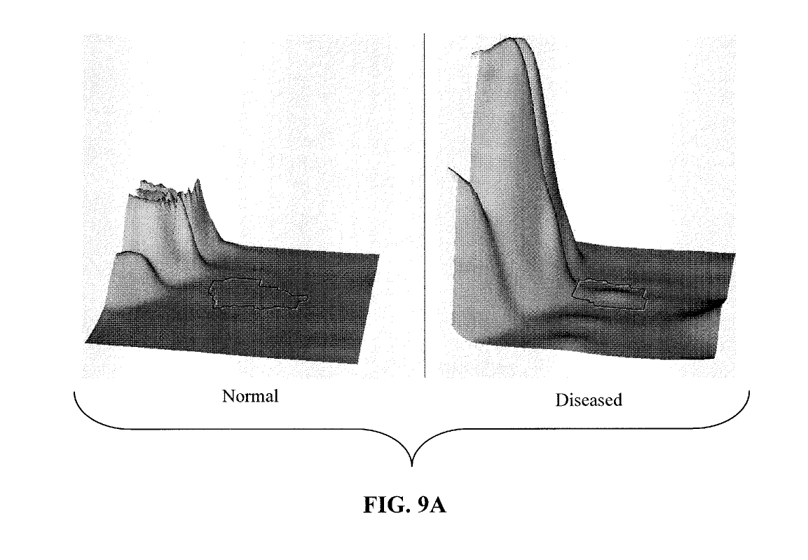

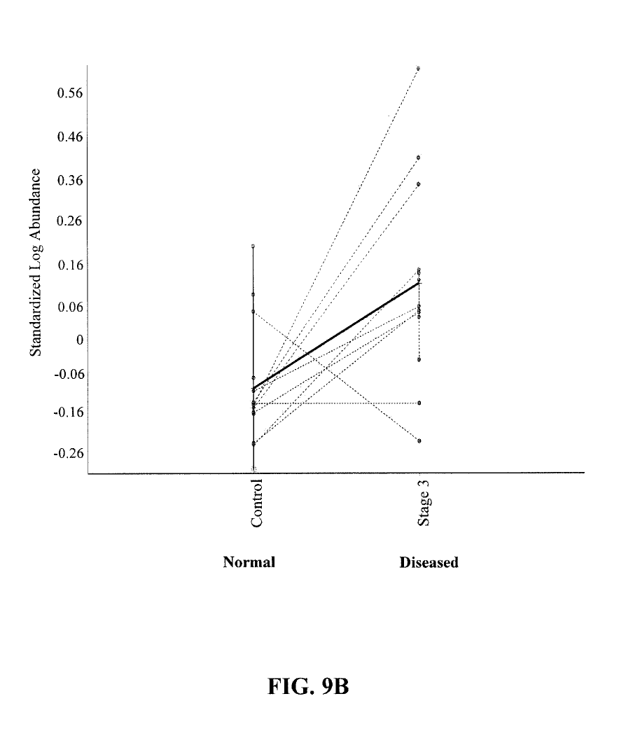

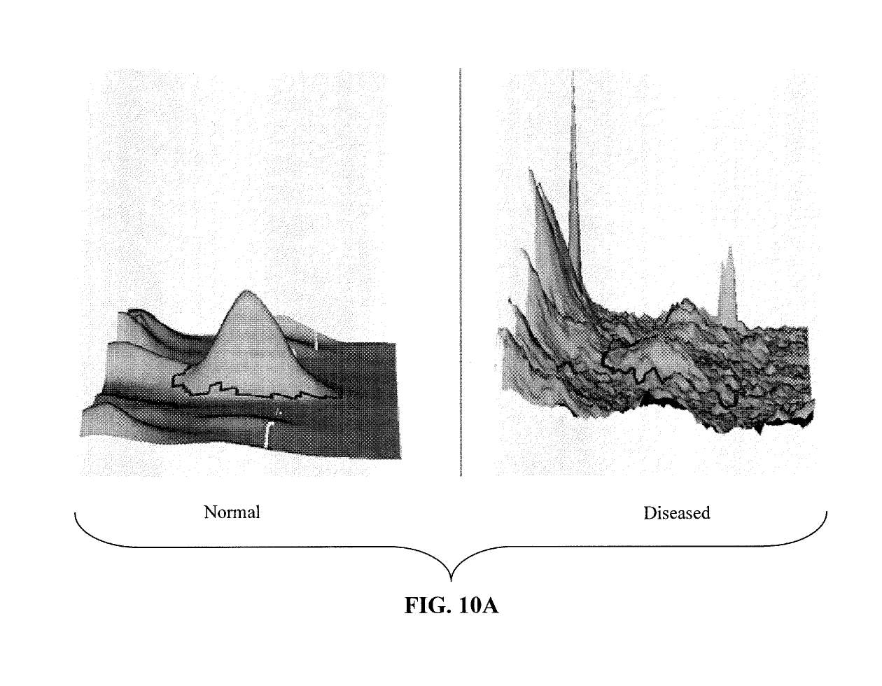

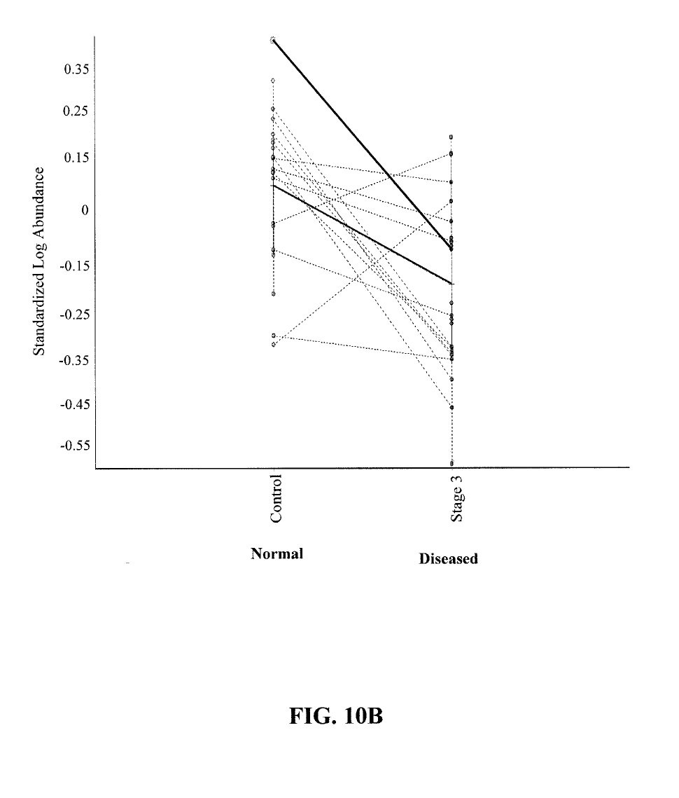

A total of 102 plasma samples (51 from donors with disease and 51 from donors without disease) have been analyzed by 2D-DIGE to identify proteins that show differential expression (see FIGS. 5-10). The study was performed in three stages:

The first stage was a proof-of-concept step in which 12 samples were analyzed with the aim of determining if the 2D-DIGE technology was sensitive enough to detect differences between “Test” (with disease) and “reference” (without disease) samples.

The second stage involved the analysis of 50 samples and identification of differentially expressed proteins by mass spectrometry.

The third stage involved the analysis of 52 more samples, identification of differentially expressed proteins and comparison of results from the first and second stages.

Out of all the candidate protein spots, five proteins with statistically significant difference (p≦0.05) in abundance between “test” and “reference” samples, and that appeared in at least 50% of the gel samples in the second and third stages were identified. Candidate proteins can be further studied, e.g., by western analysis and, if validated, a high throughput assay developed.

A total of 20 samples (10 from donors with disease and 10 from donors without disease) were also analyzed by SELDI-TOF MS in an attempt to identify distinguishing proteomic patterns that can be used for donor stratification. Distinguishing patterns in plasma samples that allowed the accurate clustering of samples into two major groups; “test” and “reference” were detected. The two groups were segregated using the hierarchical agglomerative clustering algorithm and by Principal Component Analysis (PCA).

### Example 2

The inventors compared the proteins in a series of 79 preoperative blood samples from women with ovarian cancer with samples from 81 healthy women. Of the 79 samples, 28 came from women with early stage disease. All samples were analyzed using the proteomics method known as 2D-DIGE. This enables one to compare proteins (and their levels) in two samples, and also provides the ability to identify the specific proteins that differ.

Analysis of proteomic data identified statistically significant differences in the levels of proteins between: 1) advanced stage cases and healthy controls (14 proteins, see Table 2), and 2) early stage cases and healthy controls (8 proteins, see Table 2). Of note, two proteins were identified in both analyses (alpha-1 microglobin/bikunin precursor and haptoglobin). Review of the current literature confirms the biologic plausibility of all of the identified proteins playing a role in ovarian cancer pathogenesis. Few reports exist regarding proteomics investigations in early stage samples, and the inventors believe that our data from the comparison of early stage with healthy control samples may prove particularly valuable.

Table 2 is a table comprising identified proteins that are present at different levels in the blood of women with ovarian cancer compared with healthy women.

| | | Accession | | | Av. |
|---|---|---|---|---|---|
| SN | Target | # | MW | pH | Ratio |
| | Normal vs. Late Stage | | | | |
| 1 | Ceruloplasmin | 1620909 | 122.2 | 5.44 | 1.95 |
| 2 | Keratin 10 (Cytokeratin 10) | 71528 | 59.5 | 5.17 | 1.99 |
| 3 | Haptoglobin | 4826762 | 45.2 | 6.13 | 1.52 |
| 4 | GTP Binding Protein | A44393 | 66.0 | 8.16 | 1.52 |
| 5 | Leucine-rich alpha-2-Glycoprotein | 720559 | 38.2 | 6.45 | 2.46 |
| 6 | Alpha-l-Acid Glycoprotein | 1197209 | 23.5 | 4.93 | 2.04 |
| 7 | HP Protein (Histidine) | 47124562 | 31.4 | 8.48 | 2.42 |
| 8 | Alpha-1-Anti Proteinase (Clade A) | 50363219 | 46.7 | 5.37 | 2.27 |
| 9 | Immunoglobulin Heavy Chain | 10334547 | 41.3 | 8.36 | −1.67 |
| 10 | Alpha-l-Microglobin/Bikunin | 4502067 | 39.0 | 5.95 | 1.68 |
| | Precursor | | | | |
| 11 | Poly Ubiquitin C | 21361091 | 8.6 | 6.79 | 4.3 |
| 12 | Human Cystatin A | 15988456 | 11 | 5.38 | 4.3 |
| 13 | Dermicidin precursor, AIDD | 16751921 | 11.3 | 6.09 | 4.3 |
| | protein | | | | |
| 14 | Hemoglobin Delta Chain | 122467 | 15.1 | 8.73 | 5.22 |
| | Normal vs. Early Stage | | | | |
| 15 | Hemopexin | 11321561 | 51.7 | 6.55 | 1.97 |
| 16 | Human IgG1 | 8569503 | 23.5 | 7.81 | −1.31 |
| 17 | Haptoglobin | 3337390 | 38.2 | 6.14 | 1.59 |
| 18 | Serine/Cysteine Protease inhibitor | 15080499 | 46.7 | 5.36 | −1.54 |
| 19 | Clusterin | 42716297 | 57.8 | 6.25 | −2.07 |
| 20 | Ficolin | 13124185 | 32.9 | 6.2 | −1.26 |
| 21 | Alpha-l-Microglobin/Bikunin | 4502067 | 39.0 | 5.95 | −1.67 |
| | Precursor | | | | |
| 22 | Amyloid P Component | 30582339 | 25.4 | 6.1 | −1.56 |

Although other reports exist proposing various proteins and protein panels as biomarkers of ovarian cancer, none has been sufficiently validated to receive approval as a clinical test. Additionally, spurious results of proteomics analyses are frequently reported due to poor experimental design and quality control, particularly in sample handling. All of our analyses involved careful and standardized sample collection, handling and processing. Moreover, the proteomic methodology utilized in this study is considered superior to many of the techniques used in published reports which did not allow specific identification of the individual proteins differentially measured. Notably, the single proteomic “fingerprint” that approached successful commercialization (Ovacheck) utilized surface-enhanced laser desorption/ionization (SELDI) technology, which is unable to characterize specific proteins and is thus considered inferior to the matrix-assisted laser desorption/ionization (MALDI) technology the inventors utilized. This fact and subsequently weaknesses in the statistical analysis approaches taken in the Ovacheck test resulted in its failure to achieve clinical application.

All patents, patent applications, provisional applications, and publications referred to or cited herein, supra or infra, are incorporated by reference in their entirety, including all figures and tables, to the extent they are not inconsistent with the explicit teachings of this specification.

It should be understood that the examples and embodiments described herein are for illustrative purposes only and that various modifications or changes in light thereof will be suggested to persons skilled in the art and are to be included within the spirit and purview of this application. In addition, any elements or limitations of any invention or embodiment thereof disclosed herein can be combined with any and/or all other elements or limitations (individually or in any combination) or any other invention or embodiment thereof disclosed herein, and all such combinations are contemplated with the scope of the invention without limitation thereto.

### REFERENCES

U.S. Pat. No. 5,312,922
U.S. Pat. No. 4,582,789
U.S. Pat. No. 4,617,261
U.S. Pat. No. 4,683,195
U.S. Pat. No. 3,654,090
U.S. Pat. No. 3,850,752
U.S. Pat. No. 4,016,043
Imman (1974) Methods In Enzymology, Vol. 34, Affinity Techniques, Enzyme Purification: Part B, Jakoby and Wichek (eds.), Academic Press, New York, p. 30.
Wilchek and Bayer (1988) “The Avidin-Biotin Complex in Bioanalytical Applications” Anal. Biochem. 171:1-32
Christopoulos, T. K., Diamandis, E. P. (1992) Anal. Chem. 64:342-346.
Saiki et al. (1985) Science, 230:1350
Scharf et al. (1986) Science, 233:1076
Anderson, L., Hunter, C. L. (2006) “Quantitative mass spectrometric multiple reaction monitoring assays for major plasma proteins” Mol. Cell. Proteomics 5:573-588.
Kuhn, E. et al. (2004) “Quantification of C-reactive protein in the serum of patients with rheumatoid arthritis using multiple reaction monitoring mass spectrometry and ¹³C-labeled peptide standards” Proteomics 4:1175-1186.
Liao, H. et al. (2004) “Use of mass spectrometry to identify protein biomarkers of disease severity in the synovial fluid and serum of patients with rheumatoid arthritis” Arthritis & Rheumatism 50(12):3792-3803.
Anderson, N. L. et al. (2004) “Mass spectrometric quantitation of peptides and proteins using stable isotope standards and capture by anti-peptide antibodies (SISCAPA)” J Proteome Res. 3:235-244.
Sites, D. P. et al. (1982) Chapter 22 of the 4^{th} Edition of Basic and Clinical Immunology, published by Lange Medical Publications of Los Altos, Calif.
Roitt, I., Brostoff, J., Male, D. (1998) Immunology, London: Mosby, 5th ed.
Janeway, C. A., Travers, P. (1994) Immunobiology: Immune System in Health and Disease, Oxford: Blackwell Sci. Pub.

We claim:

1. A method for detecting, diagnosing, and/or monitoring therapy for ovarian cancer in a subject, said method comprising analyzing a biological sample from the subject for the presence or absence, or the amount or concentration, of one or more proteins whose expression is associated with ovarian cancer, whereby ovarian cancer can be detected, diagnosed, and/or monitored.

2. The method according to claim 1, wherein said biological sample is selected from the group consisting of whole blood, blood plasma, serum, urine, tears, saliva, sputum, exhaled breath, nasal secretions, pharyngeal exudates, bronchoalveolar lavage, tracheal aspirations, interstitial fluid, lymph fluid, meningeal fluid, amniotic fluid, glandular fluid, feces, perspiration, mucous, vaginal or urethral secretion, cerebrospinal fluid, and transdermal exudate.

3. The method according to claim 1, wherein said biological sample is whole blood, plasma, or serum.

4. The method according to claim 1, wherein said ovarian cancer is early stage ovarian cancer.

5. The method according to claim 1, wherein said ovarian cancer is late stage ovarian cancer.

6. The method according to claim 1, wherein said one or more proteins is ceruloplasmin, keratin 10 (cytokeratin 10), haptoglobin, GTP binding protein, leucine-rich alpha-2-glycoprotein, alpha-1-acid glycoprotein, HP protein (histidine), alpha-1-anti proteinase (Clade A), immunoglobulin heavy chain, alpha-1-microglobin/bikunin precursor, poly ubiquitin C, human cystatin A, dermicidin precursor, AIDD protein, hemoglobin delta chain, hemopexin, human IgG1, serine/cysteine protease inhibitor, clusterin, ficolin, or amyloid P component, or any combination thereof.

7. The method according to claim 1, wherein said biological sample is analyzed for ceruloplasmin, keratin 10, leucine-rich alpha-2-glycoprotein, and immunoglobulin heavy chain proteins, or any combination thereof.

8. The method according to claim 1, wherein said biological sample is analyzed for haptoglobin and alpha-1-microglobin/bikunin precursor protein.

9. The method according to claim 1, wherein the subject is a human.

10. The method according to claim 1, wherein the subject is an animal other than a human.

11. The method according to claim 1, wherein said one or more proteins are detected using one or more antibodies that specifically bind to said one or more proteins.

12. The method according to claim 1, wherein said one or more proteins are analyzed using a quantitative ELISA.

13. The method according to claim 1, wherein said one or more proteins are detected using a mass spectrometry method and/or a chromatographic method.

14. The method according to claim 13, wherein said mass spectrometry method is quantitative mass spectrometric multiple reaction monitoring method.

15. The method according to claim 1, wherein said method further comprises identifying one or more proteins that are expressed at higher or lower levels in a subject having ovarian cancer as compared to a subject not having ovarian cancer.

16. The method according to claim 1, wherein the level of said one or more proteins is compared to a control reference standard of the same one or more proteins to determine whether the level of said one or more proteins in said sample is greater or lesser than said control reference standard.

17. The method according to claim 1, wherein said biological sample is analyzed for ceruloplasmin, keratin 10 (cytokeratin 10), GTP binding protein, leucine-rich alpha-2-glycoprotein, alpha-1-acid glycoprotein, HP protein (histidine), alpha-1-anti proteinase (Clade A), immunoglobulin heavy chain, poly ubiquitin C, human cystatin A, dermicidin precursor, AIDD protein, and hemoglobin delta chain, or any combination thereof.

18. The method according to claim 1, wherein said biological sample is analyzed for hemopexin, human IgG1, haptoglobulin, serine/cysteine protease inhibitor, clusterin, ficolin, alpha-1-microglobin/bikunin precursor, and amyloid P component, or any combination thereof.

19. The method according to claim 1, wherein said biological sample is analyzed for leucine-rich alpha-2-glycoprotein, alpha-1-acid glycoprotein, HP protein (histidine), alpha-1-anti proteinase (Clade A), poly ubiquitin C, human cystatin A, dermicidin precursor, AIDD protein, hemoglobin delta chain, and clusterin, or any combination thereof.

20. The method according to claim 1, further comprising comparing the presence or absence, or the amount or concentration, of said one or more proteins in said sample with the presence or absence, or the amount or concentration, of said one or more proteins in a sample from a subject without ovarian cancer.
